(19) United States
(12) Patent Application Publication
(10) Pub. No.: US 2008/0097580 A1
(43) Pub. Date: Apr. 24, 2008

(54) Morphological structures for polymeric drug delivery devices

(76) Inventor: Vipul Bhupendra Dave, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/585,065

(22) Filed: Oct. 23, 2006

(51) Int. Cl. A61F 2/90; A61F 2/94

(52) U.S. Cl. 623/1.16; 623/1.42; 623/1.49

(57) ABSTRACT

An implantable medical apparatus includes a structure formed from bioabsorbable polymers. The apparatus also contains a therapeutic agent dispersed throughout the structure or coated on the structure in such a manner as to elute the therapeutic agent when implanted in an anatomical conduit. The apparatus can be constructed from a blend of polymers and other agents. The apparatus is implanted into the conduit by expansion with a balloon or some other expandable means. The morphology or arrangement of the polymeric structure ensures that the device maintains its shape characteristics to ensure proper modeling of the vessel. In particular, the crystallinity of the polymeric structure is adjusted so as to resist recoil.

### FIELD OF THE INVENTION

The present invention relates to intraluminal polymeric devices, such as intraluminal polymeric drug eluting stents, formed from polymers blended with various additives, modifiers and active agents that enhance and optimize the performance of the medical devices constructed therefrom. In particular, these polymeric devices may have morphological variations due to the application of stress and post processing steps, respectively.

### BACKGROUND OF THE INVENTION

Currently manufactured polymeric medical devices do not adequately provide sufficient tailoring of the properties of the material forming the device to the desired mechanical behavior of the device under clinically relevant in-vivo loading conditions. For example, a polymeric stent that has been implanted in a vessel exhibits recoil causing the stent to lose apposition to the vessel wall. It is crucial for an intraluminal device such as a stent to exhibit certain characteristics, including maintaining vessel patency through an acute and/or chronic outward force that will help to remodel the vessel to its intended luminal diameter, preventing excessive radial recoil upon deployment, exhibiting sufficient fatigue resistance and exhibiting sufficient ductility so as to provide adequate coverage over the full range of intended expansion diameters.

Various mechanical approaches have been attempted to prevent recoil in an intraluminal device. For example, stents can be provided with locking mechanisms or can be constructed from a material that plastically deforms. These approaches exhibit several shortcomings. Locking mechanisms are difficult to manufacture and complicate deployment of the intraluminal device within the vessel wall. In addition, a locking mechanism may malfunction and create difficulty in producing a device with differing expansion ratios.

It is also desired to manufacture intraluminal devices such that they are bioabsorbable. This allows for later re-intervention at a site without the need to navigate around and/or remove a non-bioabsorbable device. Bioabsorption is accomplished by utilizing certain materials to construct the intraluminal devices. Locking mechanisms and other mechanical devices can be difficult to construct from these materials. Instead, it is preferable to construct the device from a material composition that will have sufficient ductility to create the desired mechanical performance for the devices such as low recoil, high radial stiffness and optimal absorption characteristics.

Intraluminal devices often are coated with a therapeutic drug that further ensures proper modeling of a conduit, such as a vessel, by preventing restenosis or neointimal hyperplasia. Polymeric devices improve the delivery of the therapeutic drug and can be formed such that the drug is dispersed within the polymer matrix. The process for creating a heterogeneous mixture of therapeutic drug and polymer present several difficulties. For example, the temperature during creation of the polymer drug mixture must be maintained at a level that will not degrade the efficacy of the drug. Finally, in order to place the drug within the polymer matrix a solvent may be employed. The removal of the solvent causes the polymer to assume a structure that affects performance of the device.

Currently, there is no device constructed from a material having preferred morphological characteristics to ensure optimal performance. The present invention is designed to address this need.

### SUMMARY OF THE INVENTION

The present invention generally comprises an intraluminal device constructed from a unique polymeric composition having properties that allow the implantable medical device to enhance performance characteristics such as high radial stiffness, minimized recoil values, and improved flexibility. More particularly, the molecular structure of the composition facilitates the design of medical devices with a wide range of geometries that are adaptable to various loading conditions. The composition and the medical devices constructed may be utilized for any number of medical applications, including vessel patency devices, such as vascular stents, biliary stents, ureter stents, vessel occlusion devices such as atrial septal and ventricular septal occluders, patent foramen ovale occluders and orthopedic devices such as fixation devices.

Forming a medical device, such as an implantable or intraluminal medical device, from bioabsorbable polymers must be accomplished in such a manner as to insure that the device maintains patency when implanted into a vessel or other conduit within a body. For example, a polymeric stent is typically implanted into a vessel by expansion with a balloon or some other expandable means. It is crucial to ensure that the stent impinges upon the inner wall of the vessel. After expansion, however, the polymer stent will experience shrinkage or recoil that causes it to lose apposition. Thus, it is desirable to minimize recoil. The morphology or arrangement of the polymeric structure is optimized in the present invention to reduce recoil.

It is also a feature of the present invention to provide a device constructed from a polymer that is mixed with a therapeutic drug that is eluted from the device upon implantation. Typically, the drug is not soluble within the polymer matrix. Instead, a solvent is utilized to form the mixture and later removed. Upon removal, the drug remains dispersed within the polymeric structure. The process utilized in removing the solvent, however, results in the crystallization of the polymer and the drug. It is desirable to crystallize the drug. As stated above, polymer crystallization affects the performance of the device constructed there from.

The present invention reduces the crystallinity of the polymer composition while preserving the crystallinity and efficacy of the therapeutic drug by creating a generally amorphous polymeric structure. Amorphous polymeric structures experience a higher level of viscous deformation at all temperatures. This type of deformation tends to be permanent in nature leading to lower recoil values of the device constructed therefrom. The present invention obtains the optimal amorphous structure by heating the polymer to at least the melting transition temperature followed by rapid quenching to prevent any recrystallization of the polymer. The polymer selected typically has a melt transition temperature below the melt transition temperature for the drug mixed therewith. Thus, the drug maintains its crystallinity and experiences minimal degradation during this process.

The devices of the present invention may also be formed from blends of polymeric materials, blends of polymeric materials and plasticizers, blends of polymeric materials and therapeutic agents, blends of polymeric materials and radiopaque agents, blends of polymeric materials with both therapeutic and radiopaque agents, blends of polymeric materials with plasticizers and therapeutic agents, blends of polymeric materials with plasticizers and radiopaque agents, blends of polymeric materials with plasticizers, therapeutic agents and radiopaque agents, and/or any combination thereof. By blending materials with different properties, a resultant material may have the beneficial characteristics of each independent material. Stiff and brittle materials may be blended with soft and elastomeric materials to create a stiff and tough material. In addition, by blending either or both therapeutic agents and radiopaque agents together with the other materials, higher concentrations of these materials may be achieved as well as a more homogeneous dispersion. Various methods for producing these blends include solvent and melt processing techniques.

For example, and in accordance with an aspect of the present invention, an implantable intraluminal medical device comprises a structure formed from at least one generally amorphous polymer, and at least one therapeutic agent dispersed throughout the at least one polymer in a concentration of up to fifty percent. By further way of example, an implantable intraluminal medical device in accordance with the present invention comprises a structure formed from a first material, and a coating layer affixed to the first material, the coating layer including at least one therapeutic agent dispersed throughout a polymeric material in a concentration of up to fifty percent.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 is a side view of a medical device fabricated from materials in accordance with the present invention.

FIG. 1A is a planar representation of an exemplary intraluminal device fabricated from materials in accordance with the present invention.

FIG. 2 is a schematic representation of a stress-strain curve of a stiff and brittle material and a plasticized material in accordance with the present invention.

FIG. 3 is a schematic representation of a stress-strain curve of a stiff and brittle material, a soft and elastomeric material and a blend of the stiff and elastomeric material in accordance with the present invention.

FIG. 4 is a graphic summarization of recoil values as a function of time for stents prepared from melt processed polymer compositions with differing crystallinity.

FIG. 5 is a summary of Differential Scanning Calorimetry (DSC) scans of solution processed polymer compositions.

FIG. 6 is a graphic summarization of recoil values as a function of time for stents prepared from solution processed polymer composition with differing crystallinity.

### DETAILED DESCRIPTION OF THE INVENTION

Implantable medical devices may be fabricated from any number of suitable biocompatible materials, including materials such as polymeric materials. The internal structure of these polymeric materials may be altered utilizing mechanical and/or chemical manipulation. These modifications may be utilized to create devices having specific characteristics such as crystalline and amorphous morphology and orientation.

In accordance with the present invention, implantable medical devices may be fabricated from any number of biocompatible polymeric materials. These polymeric materials may be non-degradable, biodegradable and/or bioabsorbable. These polymeric materials may be formed from single polymers, blends of polymers and blends of polymers and plasticizers. In addition, other agents such as drugs and/or radiopaque agents may be blended with the polymeric materials or affixed or otherwise added thereto. A number of chemical and/or physical processes may be utilized to alter the chemical and physical properties of the materials and ultimately the final devices.

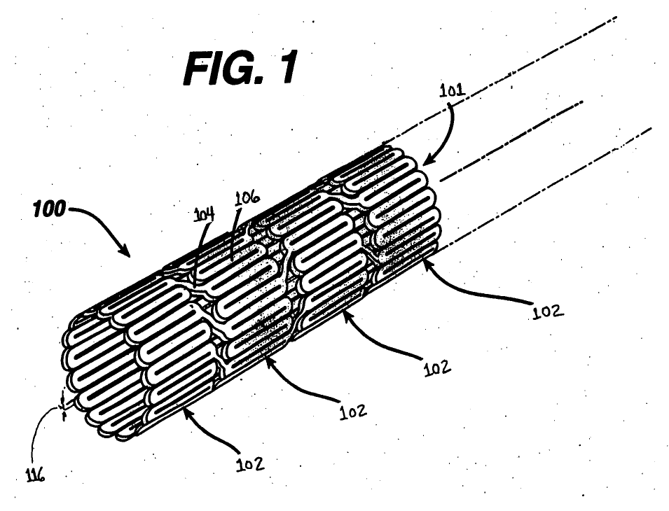

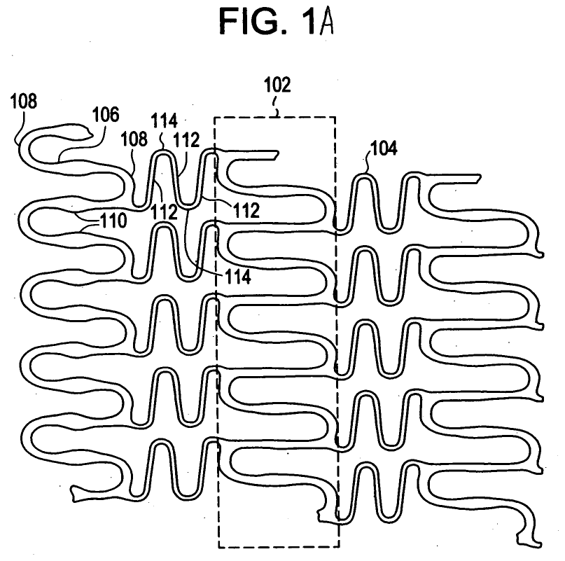

One example of a medical device that can be manufactured from the materials describe above is a stent. A stent is commonly used as a tubular structure left inside the lumen of a duct to relieve an obstruction. Referring to FIG. 1, there is illustrated a side view of a stent 100 that is manufactured in accordance with the present invention. As shown in FIG. 1A, the stent 100 comprises a plurality of hoop components 102 interconnected by a plurality of flexible connectors 104. The hoop components 102 are formed as a continuous series of substantially longitudinally or axially oriented radial strut members 106 and alternating substantially circumferentially oriented radial arc members 108. Although shown in planar view, the hoop components 102 are essentially ring members that are linked together by the flexible connectors 104 to form a substantially tubular stent structure. The combination of radial strut members 106 and alternating radial arc members 108 form a generally sinusoidal pattern but other patterns may be formed such as a zig-zag by connecting radial strut members directly together. Although the hoop components 102 may be designed with any number of features and assume any number of configurations, in the exemplary embodiment, the radial strut members 106 are wider in their central regions 110. This design feature may be utilized for a number of purposes, including, increased surface area that assists in delivering a therapeutic agent, as discussed in greater detail below.

The flexible connectors 104 are formed from a continuous series of flexible strut members 112 and alternating flexible arc members 114. The flexible connectors 104, as described above, connect adjacent hoop components 102 together. In this exemplary embodiment, the flexible connectors 104 have a substantially N-shape with one end being connected to a radial arc member on one hoop component and the other end being connected to a radial arc member on an adjacent hoop component. As with the hoop components 102, the flexible connectors 104 may comprise any number of design features and any number of configurations. In the exemplary embodiment, the ends of the flexible connectors 104 are connected to different portions of the radial arc members of adjacent hoop components for ease of nesting during crimping of the stent. It is interesting to note that with this exemplary configuration, the radial arcs on adjacent hoop components are slightly out of phase, while the radial arcs on every other hoop component are substantially in phase. In addition, it is important to note that not every radial arc on each hoop component need be connected to every radial arc on the adjacent hoop component.

A wide variety of designs may be utilized for the flexible connectors or connectors in an intraluminal scaffold or stent. For example, in the design described above, the connector comprises two elements, substantially longitudinally oriented strut members and flexible arc members. In alternate designs, however, the connectors may comprise only a substantially longitudinally oriented strut member and no flexible arc member or a flexible arc connector and no substantially longitudinally oriented strut member.

The substantially tubular structure of the stent 100 provides either temporary or permanent scaffolding for maintaining patency of body conduits, such as arteries. The stent 100 comprises a luminal surface and an abluminal surface. The distance between the two surfaces defines the wall thickness 116. The stent 100 is usually inserted into the lumen of a body conduit in a non-expanded form and are then expanded autonomously (or with the aid of a second device) in situ. When used in coronary artery procedures for relieving stenosis, stents are placed percutaneously through the femoral artery. In this type of procedure, the stent 100 is delivered on a catheter and are either self-expanding or, in the majority of cases, expanded by a balloon.

It should be understood that the present invention may be utilized not only in connection with an expandable intraluminal vascular graft for expanding partially occluded segments of a blood vessel, duct or body passageways, such as within an organ, but may so be utilized for many other purposes as an expandable prosthesis for many other types of body passageways. For example, expandable prostheses may also be used for such purposes as: (1) supportive graft placement within blocked arteries opened by transluminal recanalization, but which are likely to collapse in the absence of internal support; (2) similar use following catheter passage through mediastinal and other veins occluded by inoperable cancers; (3) reinforcement of catheter created intrahepatic communications between portal and hepatic veins in patients suffering from portal hypertension; (4) supportive graft placement of narrowing of the esophagus, the intestine, the ureters, the uretha, etc.; (5) intraluminally bypassing a defect such as an aneurysm or blockage within a vessel or organ; and (6) supportive graft reinforcement of reopened and previously obstructed bile ducts. Accordingly, use of the term “prosthesis” encompasses the foregoing usages within various types of body passageways, and the use of the term “intraluminal graft” encompasses use for expanding the lumen of a body passageway. Further in this regard, the term “body passageway” encompasses any lumen or duct within the human body, such as those previously described, as well as any vein, artery, or blood vessel within the human vascular system.

The components of the stent lattice, i.e. hoops, loops, struts and flexible links, have drugcoatings, drug and polymer coating combinations, and/or drug dispersed throughout the polymer that is used to fabricate the stent that are used to deliver drugs, i.e. therapeutic and/or pharmaceutical agents including: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP)II_{b}IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); antiinflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF) platelet derived growth factor (PDGF), erythropoetin; angiotensin receptor blocker; nitric oxide donors; anti-sense oligionucleotides and combinations thereof; cell cycle inhibitors, mTOR inhibitors, and growth factor signal transduction kinase inhibitors. It is important to note that one or more of the lattice components (e.g. hoops, loops, struts and flexible links) are coated with one or more of the drug coatings or drug and polymer coating combinations. Additionally, as mentioned above, the stent 100 is alternatively made of a polymer material itself such as a biodegradable material capable of containing and eluting one or more drugs, in any combination, in accordance with a specific or desired drug release profile.

The method of utilizing the stent 100 according to the present invention includes first identifying a location, for example, a site within the vessel in a patient's body for deployment of the stent 100. Upon identifying the desired deployment location, for example a stenotic lesion or vulnerable plaque site, a delivery device, such as a catheter carrying the stent 100 crimped to a distal end of the catheter such that the stent 100 is in its closed configuration, is inserted within the vessel in the patient's body. The catheter is used to traverse the vessel until reaching the desired location (site) wherein the distal end of the catheter is positioned at the desired location (site), for instance the lesion, within the vessel. At this point, the stent 100 is deployed to its open configuration by expanding the stent 100 such as by inflation if the stent 100 is a balloon expandable stent or by uncovering or release of the stent 100 if the stent 100 is a self-expanding (crush recoverable) type stent. If a cover is utilized to further protect and secure the stent 100 to the catheter distal end when the stent 100 is a self-expanding stent, the cover is removed from the distal end of the catheter prior to expansion of the stent 100, for instance, through use of an expandable member such as an inflatable balloon. Upon expanding the stent 100 to its open configuration, the expandable member (balloon) is then collapsed, for instance through deflation of the expandable member, whereby the catheter is removed from the deployment site of the vessel and patient's body altogether.

The stent 100 can be delivered by balloon expansion; self-expansion; or a balloon assist self expansion delivery system. The benefit of using the combination system is that stent 100 is not crimped to lower profiles and upon deployment the stent will self expand to a certain value and can be further expanded to the desired dimension by balloon expansion.

Once the stent is in place, the conduit should assume a modeled shape that ensures the proper flow of fluids there through. Nonetheless, additional procedures may be required at other locations downstream from the location where the stent has been placed. In performing these procedures, the presence of pre-placed stents must be taken into consideration as the stent must be passed through to reach the downstream site. Thus, it is advantageous to manufacture a stent from a biodegradable substance, such as a polymer. Polymeric stents, however, may not prevent restenosis as a result of elastic recoil of the polymeric materials. As mentioned previously, the unique design of the stent 100 allows for a wide array of materials, not previously used with prior art stents, to be used with the stent 100 in accordance with the present invention. These include materials normally prone to crushing, deformation or recoil upon deployment of the stent. These materials include plastics and polymers to include biodegradable polymers such as drug eluting polymers.

In general, the stent 100 can be constructed from biodegradable or bioabsorbable polymer compositions. The type of polymers used can degrade via different mechanisms such as bulk or surface erosion. Bulk erodible polymers include aliphatic polyesters such poly (lactic acid); poly (glycolic acid); poly (caprolactone); poly (p-dioxanone) and poly (trimethylene carbonate); and their copolymers and blends. Other polymers can include amino acid derived polymers; phosphorous containing polymers [e.g., poly (phosphoesters)] and poly (ester amide). Surface erodible polymers include polyanhydrides and polyorthoesters. The stent 100 can be made from combinations of bulk and surface erodible polymers to control the degradation mechanism of the stent. For example, the regions that are under high stress can be made from a polymer that will retain strength for longer periods of time, as these will degrade earlier than other regions with low stress. The selection of the polymers will determine the absorption of stents 100 that can be very short (few weeks) and long (weeks to months).

The bioabsorbable compositions to prepare the stent 100 will also include drug and radiopaque materials. The amount of drug can range from about 1 to 30 percent as an example, although the amount of drug loading can comprise any desired percentage. The stent 100 will carry more drug than a polymer-coated stent. The drug will release by diffusion and during degradation of the stent 100. The amount of drug release will be for a longer period of time to treat local and diffuse lesions; and for regional delivery for arterial branches to treat diseases such as vulnerable plaque. Radiopaque additives can include barium sulfate and bismuth subcarbonate and the amount can be from 5 to 30 percent as an example. Other radiopaque materials include gold particles and iodine compounds. The particle size of these radiopaque materials can vary from nanometers to microns. The benefit of small particle size is to avoid any reduction in the mechanical properties and to improve the toughness values of the devices. Upon polymer absorption, small particles will also not have any adverse effects on surrounding tissues.

The polymeric tubes used to prepare bioabsorbable stents 100 can be fabricated either by melt or solvent processing. The preferred method will be solvent processing, especially for the stents that will contain drug. These tubes can be converted to the desired design by excimer laser processing. Other methods to fabricate the tubes from which the stent is crafted can be injection molding using supercritical fluids such as carbon dioxide.

Recoil Reduction in Polymeric Devices

In order to reduce recoil in polymeric devices, it is desirable to modify the structure of the polymer utilized in constructing the device. There are typically three different forms in which a polymer may display the mechanical properties associated with solids; namely, as a crystalline structure, as a semi-crystalline structure and/or as an amorphous structure. All polymers are not able to fully crystallize, as a high degree of molecular regularity within the polymer chains is essential for crystallization to occur. Even in polymers that do crystallize, the degree of crystallinity is generally less than one hundred percent. Within the continuum between fully crystalline and amorphous structures, there are two thermal transitions possible; namely, the crystal-liquid transition (i.e. melting point temperature, Tₘ) and the glass-liquid transition (i.e. glass transition temperature, T_{g}). In the temperature range between these two transitions there may be a mixture of orderly arranged crystals and amorphous polymer domains.

When the molecular weight of polymers is low, molecules will diffuse or move. When the molecular weight is sufficiently high then there will be entanglements between the polymer chains. These entanglements play an important role in influencing mechanical response particularly the ultimate properties due to inability of chains to provide further slippage.

Removing load from a deformed polymer does not lead to recovery of the sample to its original dimensions and is defined as permanent deformation or irrecoverable flow. This is due to the viscoelastic nature of polymers. The elastic component and its response in the polymer depend on time and temperature. The amount of permanent set may also vary with time at a given set of conditions. Long relaxation time is associated with deformed macromolecules due to chain entanglements. Main factors that influence permanent deformation are morphological conditions (amorphous and crystalline phases), presence of fillers, level of oriented amorphous material and thermal energy. The mechanical response is different when the polymer is tested below Tg and above Tg. Below Tg, the chains are frozen so only bond stretching is possible, and the material is glassy (i.e., stiff and brittle). Above Tg, the chains are flexible and free to move and change shape during deformation. For semi-crystalline polymers, crystalline regions hold the amorphous section and there is slight restriction to chain motion due to entanglements.

Permanent deformation has an inverse relationship to recoil values (both acute and chronic) for a device prepared from polymeric materials. Viscoelastic materials deforms by a combination of amorphous or viscous component and crystalline or elastic component. When a material is deformed at time t₁, there is an instantaneous elastic response followed by a retarded elastic response. When the stress is removed at time t₂, there is an instantaneous elastic recovery followed by retarded elastic response, which results in permanent deformation. The amount of deformation is time dependent and is controlled by the viscous component. If a material is highly crystalline, then there will be low permanent deformation or high recoil of the device. If a material is fully amorphous, then there will be high permanent deformation or low recoil of the device. So, in order to achieve a desirable recoil value it is necessary to control the degree of crystallinity of the polymer that is used to prepare the device.

In order to illustrate the principles of the present invention, devices were prepared from tubular materials that were made with different amount of crystallinity. The processes used to prepare them were from melt and solution processing. Typical material composition was PLGA 85/15 blended with different amount (5 to 35%) of PCL/PGA (35/65; 45/55 and 50/50), barium sulfate (5 to 30%) and sirolimus (1 to 30%).

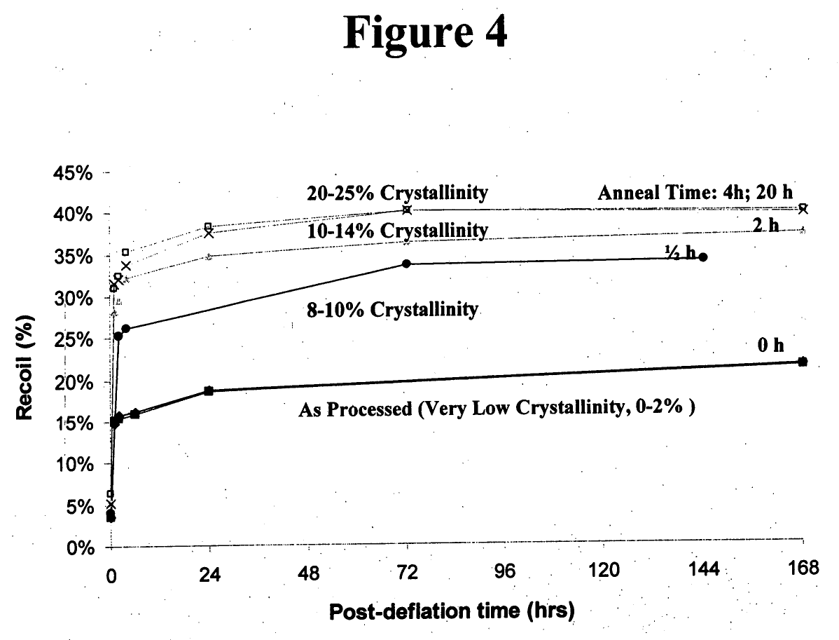

FIG. 4 is a summary of device, which in this case was a stent, recoil values for melt-processed materials prepared from PLGA 85/15 with 20% PCL/PGA (35/65) and 30% barium sulfate (BS) that were prepared with different amount of crystallinity. As-extruded materials were generally amorphous with low level of crystallinity. The tubes from which the stents were formed were annealed at 90° C. for different time (30 min; 2 h; 4 h and 20 h) to increase the amount of crystallinity from about 8 to about to 25% in the tubes. The recoil values for the stents prepared from as extruded amorphous materials increased gradually to about 15 to 20% after 7 days. It was also observed that barium sulfate did not contribute to the recoil of the stents. The recoil values increased for stents prepared from materials with increasing amount of crystallinity. It varied from about 30%, 35% to 40% for materials with about 10%, 14% and 25% crystallinity, respectively, after 7 days. This shows that there is a direct correlation between crystallinity and recoil values of the polymer stents. It is possible to further increase the recoil values by increasing the crystallinity which happens when the annealing conditions of the tubes are optimized.

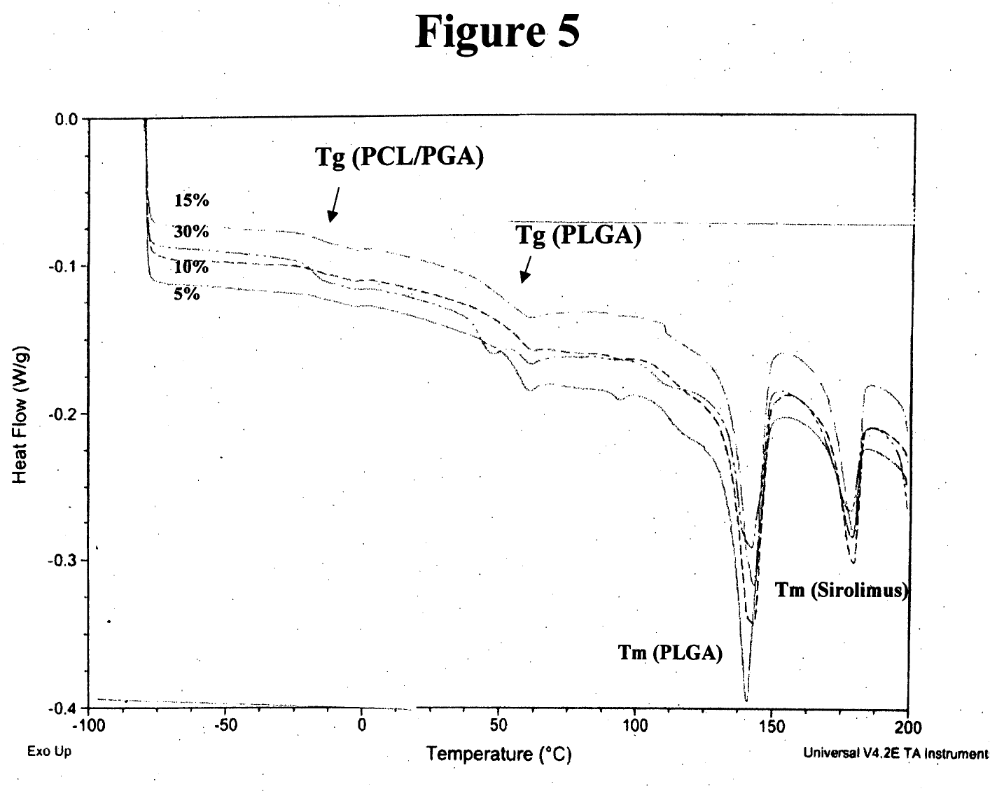

FIG. 5 shows the differential scanning calorimetry (DSC) scans of materials prepared from solvent cast process. The material composition consists of PLGA 85/15 blended with different amount of PCL/PGA (50/50) [5 to 30%], barium sulfate and sirolimus. The melt transitions at about 145° C. and 180° C. are from PLGA and sirolimus, respectively. The Tg values of PLGA and PCL/PGA are at about 50° C. and −10° C., respectively.

In order to prepare a generally amorphous structure, these materials were heated to different temperatures, for example 140 to 180° C., for different time at a given temperature, for example, 30 seconds to 40 minutes, and rapidly quenched. The resulting heat quenched materials had different amount of crystallinity, for example, 0 to 15%, with no significant affect on drug recovery. This was mainly due to the fact that the drug was in the more stable crystalline state, and the temperatures were kept below the melt transition temperatures of the drug. Some of the amorphous heat quenched materials were annealed at 90° C. for different time to increase the crystallinity values.

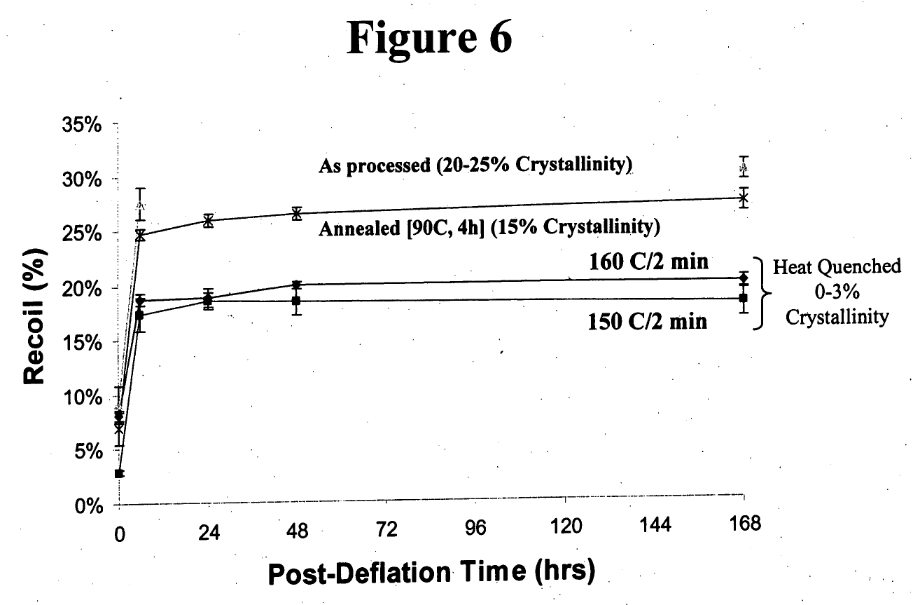

FIG. 6 is the summary of stent recoil values for solvent processed materials prepared from PLGA 85/15 with 10% PCL/PGA (50/50), 30% barium sulfate (BS) and sirolimus that were prepared with different amount of crystallinity. As processed solvent cast materials have about 20 to 25% crystallinity and exhibits about 30% recoil after 7 days. After making the materials generally amorphous (0-3% crystallinity) by using the heat-quenched process described above at different temperatures and time (for example, 150° C. and 160° C. for 2 minutes), the recoil value was about 15%-20% after 7 days. Annealing the heat-quenched materials at 90° C. for different time increased the crystallinity values. For example, a sample that was annealed for 4 h at 90° C. had about 15% crystallinity with recoil value of about 25%.

This shows that controlling the morphology (i.e., different amount of crystallinity) of the stents can have a significant affect on the recoil values. In order to have low recoil values, stents can be prepared from materials that have the desired morphology with lower levels of crystallinity and with an appropriate domain size and distribution of the polymers.

In order to provide different amount of crystallinity, different types of polymers with different molecular weight can be used to prepare the material composition. Some examples include poly(L-lactide) [PLLA], poly(DL-lactide) [PDLLA], poly(L/DL-lactide) [80/20 L/DL], poly(L-lactide/glycolide) [PLGA 95/5; PLGA 85/15; PLGA 82/18; PLGA 80/20; PLGA 75/25; PLGA 70/30], poly(DL-lactide/glycolide) [PDLGA 85/15; PDLGA 80/20; PDLGA 75/25 and PDLGA 70/30], poly(L-lactide/caprolactone), and poly(L-lactide/TMC). These materials will have different levels of crystallinity and thereby have different recoil values. Some of these materials may not require the heat quenching step as the as processed tubes will have the desired crystallinity values to achieve low recoil values.

Other Properties of Polymeric Devices

Polymeric materials may be broadly classified as synthetic, natural and/or blends thereof. Within these broad classes, the materials may be defined as biostable or biodegradable. Examples of biostable polymers include polyolefins, polyamides, polyesters, fluoropolymers, and acrylics. Examples of natural polymers include polysaccharides and proteins.

The drug delivery devices according to the systems and methods of the present invention may be disease specific, and may be designed for local or regional therapy, or a combination thereof. They may be used to treat coronary and peripheral diseases such as vulnerable plaque, restenosis, bifurcated lesions, superficial femoral artery, below the knee, saphenous vein graft, arterial tree, small and tortuous vessels, and diffused lesions. The drugs or other agents delivered by the drug delivery devices according to the systems and methods of the present invention may be one or more drugs, bio-active agents such as growth factors or other agents, or combinations thereof. The drugs or other agents of the device are ideally controllably released from the device, wherein the rate of release depends on either or both of the degradation rates of the bioabsorbable polymers comprising the device and the nature of the drugs or other agents. The rate of release can thus vary from minutes to years as desired.

Bioabsorobable and/or biodegradable polymers consist of bulk and surface erodable materials. Surface erosion polymers are typically hydrophobic with water labile linkages. Hydrolysis tends to occur fast on the surface of such surface erosion polymers with no water penetration in bulk. The initial strength of such surface erosion polymers tends to be low however, and often such surface erosion polymers are not readily available commercially. Nevertheless, examples of surface erosion polymers include polyanhydrides such as poly (carboxyphenoxy hexane-sebacic acid), poly (fumaric acid-sebacic acid), poly (carboxyphenoxy hexane-sebacic acid), poly (imide-sebacic acid)(50-50), poly (imide-carboxyphenoxy hexane) (33-67), and polyorthoesters (diketene acetal based polymers).

Bulk erosion polymers, on the other hand, are typically hydrophilic with water labile linkages. Hydrolysis of bulk erosion polymers tends to occur at more uniform rates across the polymer matrix of the device. Bulk erosion polymers exhibit superior initial strength and are readily available commercially.

Examples of bulk erosion polymers include poly (α-hydroxy esters) such as poly (lactic acid), poly (glycolic acid), poly (caprolactone), poly (p-dioxanone), poly (trimethylene carbonate), poly (oxaesters), poly (oxaamides), and their co-polymers and blends. Some commercially readily available bulk erosion polymers and their commonly associated medical applications include poly (dioxanone) [PDS® suture available from Ethicon, Inc., Somerville, N.J.], poly (glycolide) [Dexon® sutures available from United States Surgical Corporation, North Haven, Conn.], poly (lactide)-PLLA [bone repair], poly (lactide/glycolide) [Vicryl® (10/90) and Panacryl® (95/5) sutures available from Ethicon, Inc., Somerville, N.J.], poly (glycolide/caprolactone (75/25) [Monocryl® sutures available from Ethicon, Inc., Somerville, N.J.], and poly (glycolide/trimethylene carbonate) [Maxon® sutures available from United States Surgical Corporation, North Haven, Conn.].

Other bulk erosion polymers are tyrosine derived poly amino acid [examples: poly (DTH carbonates), poly (arylates), and poly (imino-carbonates)], phosphorous containing polymers [examples: poly (phosphoesters) and poly (phosphazenes)], poly (ethylene glycol) [PEG] based block co-polymers [PEG-PLA, PEG-poly (propylene glycol), PEG-poly (butylene terephthalate)], poly (α-malic acid), poly (ester amide), and polyalkanoates [examples: poly (hydroxybutyrate (HB) and poly (hydroxyvalerate) (HV) co-polymers].

Of course, the devices may be made from combinations of surface and bulk erosion polymers in order to achieve desired physical properties and to control the degradation mechanism. For example, two or more polymers may be blended in order to achieve desired physical properties and device degradation rate. Alternately, the device may be made from a bulk erosion polymer that is coated with a surface erosion polymer. The drug delivery device may be made from a bulk erosion polymer that is coated with a drug containing a surface erosion polymer. For example, the drug coating may be sufficiently thick that high drug loads may be achieved, and the bulk erosion polymer may be made sufficiently thick that the mechanical properties of the device are maintained even after all of the drug has been delivered and the surface eroded.

Shape memory polymers may also be used. Shape memory polymers are characterized as phase segregated linear block co-polymers having a hard segment and a soft segment. The hard segment is typically crystalline with a defined melting point, and the soft segment is typically amorphous with a defined glass transition temperature. The transition temperature of the soft segment is substantially less than the transition temperature of the hard segment in shape memory polymers. A shape in the shape memory polymer is memorized in the hard and soft segments of the shape memory polymer by heating and cooling techniques. Shape memory polymers may be biostable and bioabsorbable. Bioabsorbable shape memory polymers are relatively new and comprise thermoplastic and thermoset materials. Shape memory thermoset materials may include poly (caprolactone) dimethylacrylates, and shape memory thermoplastic materials may include poly (caprolactone) as the soft segment and poly (glycolide) as the hard segment.

In order to provide materials with high toughness, such as is often required for orthopedic implants, sutures, stents, grafts and other medical applications including drug delivery devices, the bioabsorbable polymeric materials may be modified to form composites or blends thereof. Such composites or blends may be achieved by changing either the chemical structure of the polymer backbone, or by creating composite structures by blending them with different polymers and plasticizers. The addition of plasticizers, which are generally low molecular weight materials, or a soft (lower glass transition temperature) miscible polymer, will depress the glass transition temperature of the matrix polymer system. In general, these additional materials that are used to modify the underlying bioabsorbable polymer should preferably be miscible with the main matrix polymer system to be effective.

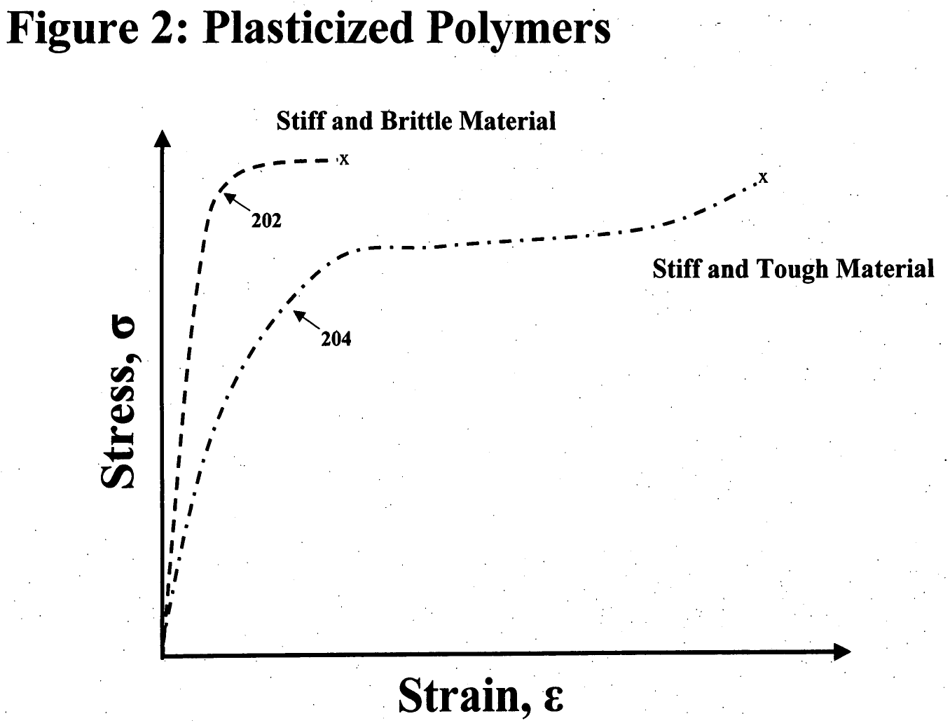

FIG. 2 is a schematic representation of the stress-strain behavior of a plasticized stiff and brittle material, represented by curve 204. The stiff and brittle polymeric material, represented by curve 202, is altered by the addition of a plasticizer. Stiff material has a higher modulus and low strain at break values with low toughness as the area under the curve is small. The addition of a plasticizer makes the stiff and brittle material a stiff and tough material. In other words, the addition of a plasticizer will lower the modulus to some extent but will increase the ultimate strain value thereby making the plasticized material tougher. As stated above, curve 204 represents the blend of a stiff and brittle polymer with a plasticizer resulting in a material with a modified stress-strain curve. The amount of change in modulus and toughness depends on the amount of plasticizer in the polymer. In general, a higher amount of plasticizer, will lower the modulus and increase the toughness values.

Plasticizers that are added to the matrix of bioabsorbable polymer materials will make the device more flexible and typically reduces the processing temperatures in case of processing materials in melt. The plasticizers are added to the bioabsorbable materials of the device prior to or during processing thereof. As a result, degradation of drugs incorporated into the bioabsorbable materials having plasticizers added thereto during processing is further minimized.

Plasticizers or mixtures thereof suitable for use in the present invention may be selected from a variety of materials including organic plasticizers and those like water that do not contain organic compounds. Organic plasticizers include but not limited to, phthalate derivatives such as dimethyl, diethyl and dibutyl phthalate; polyethylene glycols with molecular weights preferably from about 200 to 6,000, glycerol, glycols such as polypropylene, propylene, polyethylene and ethylene glycol; citrate esters such as tributyl, triethyl, triacetyl, acetyl triethyl, and acetyl tributyl citrates, surfactants such as sodium dodecyl sulfate and polyoxymethylene (20) sorbitan and polyoxyethylene (20) sorbitan monooleate, organic solvents such as 1,4-dioxane, chloroform, ethanol and isopropyl alcohol and their mixtures with other solvents such as acetone and ethyl acetate, organic acids such as acetic acid and lactic acids and their alkyl esters, bulk sweeteners such as sorbitol, mannitol, xylitol and lycasin, fats/oils such as vegetable oil, seed oil and castor oil, acetylated monoglyceride, triacetin, sucrose esters, or mixtures thereof. Preferred organic plasticizers include citrate esters; polyethylene glycols and dioxane.

Polymer blends are commonly prepared to achieve the desired final polymer properties. In accordance with the present invention, polymer blends are prepared to increase the elongation at break values or ultimate strain and thereby improving the toughness of the material that will be used to prepare vascular devices such as stents. Selection of the materials is important in order to achieve high toughness values of the matrix polymer. Matching solubility parameters and increase in free volume is important for the polymer blends to achieve the desired performance. The main difference between adding a plasticizer and a polymer to the matrix polymer is the difference in their molecular weights. As mentioned earlier, plasticizers have lower molecular weight compared to a polymeric additive. However, some low molecular weight polymers may also be used as a plasticizer. It is possible to achieve high toughness values by adding low amounts of plasticizer compared to a polymeric additive. Relatively high molecular weight material has been used as the matrix material for the present invention. For example, the molecular weight (weight average) of PLGA resins may be above 300,000 Daltons. Thermodynamically, molecular weight plays a big role in miscibility of polymer systems. There is higher miscibility between polymer and a low molecular weight additive compared to a high molecular weight additive. As mentioned earlier, the addition of a miscible polymer will lower glass transition temperature, decrease modulus and tensile strength with an increase in the toughness values.

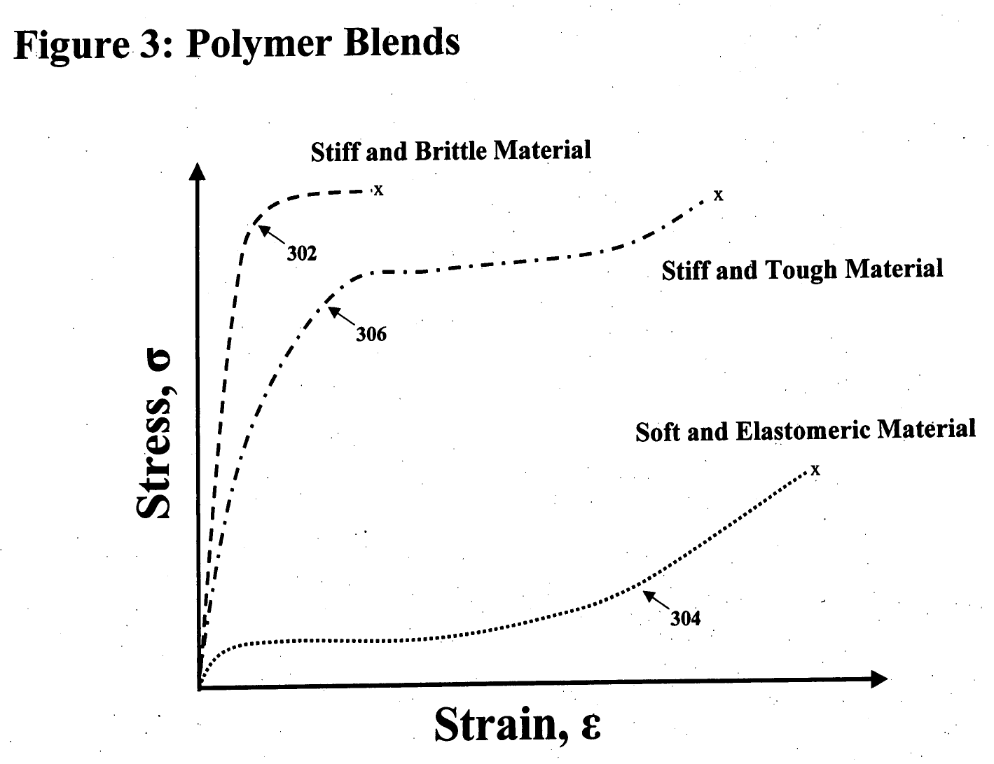

FIG. 3 is a schematic representation of the stress-strain behavior of a stiff and brittle material with high modulus and low strain at break values, i.e., low toughness, as represented by curve 302 with a soft and elastomeric material with low modulus and relatively high strain at break values, as represented by curve 304 and the resultant polymer blend prepared from these two materials, as represented by curve 306, that will provide a relatively stiff material with high ultimate strain values, i.e., high toughness. The amount of change in modulus, strength and strain at break values depends on the amount of the polymeric additive in the matrix polymer. In general, the polymers are miscible or compatible at lower levels of the additive (for example <50 percent by weight) beyond which they become phase separated and the physical properties may begin to deteriorate. However, it is important to note that it is possible to achieve desirable compatibility between the phase-separated polymers through the addition of bioabsorbable compatibilizers.

As an example of producing a composite or blended material, blending a stiff polymer such as poly (lactic acid), poly (glycolide) and poly (lactide-co-glycolide) copolymers with a soft and elastomeric polymer such as poly (caprolactone) and poly (dioxanone) tends to produce a material with high toughness and high stiffness. An elastomeric co-polymer may also be synthesized from a stiff polymer and a soft polymer in different ratios. For example, poly (glycolide) or poly (lactide) may be copolymerized with poly (caprolactone) or poly (dioxanone) to prepare poly(glycolide-co-caprolactone) or poly(glycolide-co-dioxanone) and poly(lactide-co-caprolactone) or poly(lactide-co-dioxanone) copolymers. These elastomeric copolymers may then be blended with stiff materials such as poly (lactide), poly (glycolide) and poly (lactide-co-glycolide) copolymers to produce a material with high toughness and ductility. Alternatively, terpolymers may also be prepared from different monomers to achieve desired properties. For example, poly (caprolactone-co-glycolide-co-lactide) may be prepared in different ratios.

In addition to increasing the toughness values with the addition of the soft polymers, the absorption time may also be modified. For example, the blend of PLGA with polycaprolactone will increase the total absorption time of the blended material as polycaprolactone degrades slower than PLGA. The total absorption may be reduced for PLGA by blending it with faster degrading materials such as poly (dioxanone) and their copolymers with poly (glycolide) and poly (lactide); and copolymers of poly (glycolide) such as poly (caprolactone-co-glycolide). Reinforced composites may also be prepared by blending high modulus PGA fibers or bioabsorbable particulate fillers with PLGA to form composites in the presence of the plasticizers or soft materials to improve the modulus of the final material.

Melt blends of polymers, with melting points lower than the melting point of the bioabsorbable materials in which the drugs or other bio-active agents are to be incorporated, may also be added to the bioabsorbable materials that are to comprise the device. Adding the blends of polymers having the lower melting points also helps to reduce processing temperatures and minimize degradation of the drugs or agents thereby.

It is important to note that the drug or therapeutic agent, in sufficient concentration, may be used as an additive for modifying the polymer properties. In other words, the drug or therapeutic agent may be utilized as part of the blend, rather than as a material affixed to a base material, similar to the blends described herein to achieve the desired end product properties in addition to providing a therapeutic effect.

Radiopaque materials may be added to the polymer blend from which the device is constructed to ensure visualization of the device as it is implanted in the patient. The radiopaque materials may be added directly to the matrix of bioabsorbable materials comprising the device during processing thereof resulting in fairly uniform incorporation of the radiopaque materials throughout the device. Alternately, the radiopaque materials may be added to the device in the form of a layer, a coating, a mark or band or powder at designated portions of the device depending on the geometry of the device and the process used to form the device. Coatings may be applied to the device in a variety of processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques. Such coatings sometimes have less negative impact on the physical characteristics (eg., size, weight, stiffness, flexibility) and performance of the device than do other techniques.

Preferably, the radiopaque material does not add significant stiffness to the device so that the device may readily traverse the anatomy within which it is deployed. The radiopaque material should be biocompatible with the tissue within which the device is deployed. Such biocompatibility minimizes the likelihood of undesirable tissue reactions with the device. Inert noble metals such as gold, platinum, iridium, palladium, and rhodium are well-recognized biocompatible radiopaque materials. Other radiopaque materials include barium sulfate (BaSO₄), bismuth subcarbonate [(BiO)₂CO₃] and bismuth oxide. Preferably, the radiopaque materials adhere well to the device such that peeling or delamination of the radiopaque material from the device is minimized, or ideally does not occur. Where the radiopaque materials are added to the device as metal bands, the metal bands may be crimped at designated sections of the device. Alternately, designated sections of the device may be coated with a radiopaque metal powder, whereas other portions of the device are free from the metal powder.

Delivery of Therapeutic Agents

The local delivery of therapeutic agent/therapeutic agent combinations may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by combining a drug or drugs with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the device-drug combination approach.

Devices that serve to improve the structure and function of tissue or organ may also show benefits when combined with the appropriate agent or agents. For example, improved osteointegration of orthopedic devices to enhance stabilization of the implanted device could potentially be achieved by combining it with agents such as bone-morphogenic protein. Similarly other surgical devices, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, including stents, stent-grafts and other devices for repairing aneurysims, and vascular supports could also provide enhanced patient benefit using this drug-device combination approach. Perivascular wraps may be particularly advantageous, alone or in combination with other medical devices. The perivascular wraps may supply additional drugs to a treatment site. Essentially, any other type of medical device may be coated in some fashion with a drug or drug combination, which enhances treatment over use of the singular use of the device or pharmaceutical agent.

Different drugs may be utilized as therapeutic agents, including sirolimus, heparin, everolimus, tacrolimus, paclitaxel, cladribine as well as classes of drugs such as statins. These drugs and/or agents may be hydrophilic, hydrophobic, lipophilic and/or lipophobic. The type of agent will play a role in determining the type of polymer. The amount of the drug in the coating may be varied depending on a number of factors including, the storage capacity of the coating, the drug, the concentration of the drug, the elution rate of the drug as well as a number of additional factors. The amount of drug may vary from substantially zero percent to substantially one hundred percent. Typical ranges may be from about less than one percent to about forty percent or higher. Drug distribution in the coating may be varied. The one or more drugs may be distributed in a single layer, multiple layers, single layer with a diffusion barrier or any combination thereof.

In addition to various medical devices, the coatings on these devices may be used to deliver therapeutic and pharmaceutic agents including, all the compounds described above and anti-proliferative agents, anti-throrombogenic agents, anti-restenotic agents, anti-infective agents, anti-viral agents, anti-bacterial agents, anti-fungal agnts, anti-inflammatory agents, cytostatic agents, cytotoxic agents, immunosuppressive agents, anti-microbial agents, anti-calcification agents, anti-encrustation agents, statins, hormones, anti-cancer agents, anti-coagulants, anti-migrating agents and tissue growth promoting agents.

As described herein, various drugs or agents may be incorporated into the medical device by a number of mechanisms, including blending it with the polymeric materials or affixing it to the surface of the device. Different drugs may be utilized as therapeutic agents, including sirolimus, or rapamycin, heparin, everolimus, tacrolimus, paclitaxel, cladribine as well as classes of drugs such as statins. These drugs and/or agents may be hydrophilic, hydrophobic, lipophilic and/or lipophobic.

The local delivery of drug/drug combinations from a stent has the following advantages; namely, the prevention of vessel recoil and remodeling through the scaffolding action of the stent and the prevention of multiple components of neointimal hyperplasia or restenosis as well as a reduction in inflammation and thrombosis. This local administration of drugs, agents or compounds to stented coronary arteries may also have additional therapeutic benefit. For example, higher tissue concentrations of the drugs, agents or compounds may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, anti-thrombotic drugs, agents or compounds.

A variety of drugs, agents or compounds may be utilized in combination with any number of medical devices, and in particular, with implantable medical devices such as stents and stent-grafts. Other devices such as vena cava filters and anastomosis devices may be used with coatings having drugs, agents or compounds therein or the devices themselves may be fabricated with polymeric materials that have the drugs contained therein. Any of the stents or other medical devices described herein may be utilized for local or regional drug delivery. Balloon expandable stents may be utilized in any number of vessels or conduits, and are particularly well suited for use in coronary arteries. Self-expanding stents, on the other hand, are particularly well suited for use in vessels where crush recovery is a critical factor, for example, in the carotid artery.

Any of the above-described medical devices may be utilized for the local delivery of drugs, agents and/or compounds to other areas, not immediately around the device itself. In order to avoid the potential complications associated with systemic drug delivery, the medical devices of the present invention may be utilized to deliver therapeutic agents to areas adjacent to the medical device. For example, a rapamycin coated stent may deliver the rapamycin to the tissues surrounding the stent as well as areas upstream of the stent and downstream of the stent (regional delivery). The degree of tissue penetration depends on a number of factors, including the drug, agent or compound, the concentrations of the drug and the release rate of the agent. The same holds true for coated anastomosis devices.

The amount of drugs or other agents incorporated within the drug delivery device according to the systems and methods of the present invention may range from about 0 to 99 percent (percent weight of the device). The drugs or other agents may be incorporated into the device in different ways. For example, the drugs or other agents may be coated onto the device after the device has been formed, wherein the coating is comprised of bioabsorbable polymers into which the drugs or other agents are incorporated. Alternately, the drugs or other agents may be incorporated into the matrix of bioabsorbable materials comprising the device. The drugs or agents incorporated into the matrix of bioabsorbable polymers may be in an amount the same as, or different than, the amount of drugs or agents provided in the coating techniques discussed earlier if desired. These various techniques of incorporating drugs or other agents into, or onto, the drug delivery device may also be combined to optimize performance of the device, and to help control the release of the drugs or other agents from the device.

Where the drug or agent is incorporated into the matrix of bioabsorbable polymers comprising the device, for example, the drug or agent will release by diffusion and during degradation of the device. The amount of drug or agent released by diffusion will tend to release for a longer period of time than occurs using coating techniques, and may often more effectively treat local and diffuse lesions or conditions thereof. For regional drug or agent delivery such diffusion release of the drugs or agents is effective as well. Polymer compositions and their diffusion and absorption characteristics will control drug elution profile for these devices. The drug release kinetics will be controlled by drug diffusion and polymer absorption. Initially, most of the drug will be released by diffusion from the device surfaces and bulk and will then gradually transition to drug release due to polymer absorption. There may be other factors that will also control drug release. If the polymer composition is from the same monomer units (e.g., lactide; glycolide), then the diffusion and absorption characteristics will be more uniform compared to polymers prepared from mixed monomers. Also, if there are layers of different polymers with different drug in each layer, then there will be more controlled release of drug from each layer. There is a possibility of drug present in the device until the polymer fully absorbs thus providing drug release throughout the device life cycle.

The drug delivery device according to the systems and methods of the present invention preferably retains its mechanical integrity during the active drug delivery phase of the device. After drug delivery is achieved, the structure of the device ideally disappears as a result of the bioabsorption of the materials comprising the device. The bioabsorbable materials comprising the drug delivery device are preferably biocompatible with the tissue in which the device is implanted such that tissue interaction with the device is minimized even after the device is deployed within the patient. Minimal inflammation of the tissue in which the device is deployed is likewise preferred even as degradation of the bioabsorbable materials of the device occurs. In order to provide multiple drug therapy, enriched or encapsulated drug particles or capsules may be incorporated in the polymer matrix. Some of these actives may provide different therapeutic benefits such as anti-inflammatory, anti-thrombotic; etc.

In accordance with another exemplary embodiment, the stents described herein, whether constructed from metals or polymers, may be utilized as therapeutic agents or drug delivery devices wherein the drug is affixed to the surface of the device. The devices may be coated with a biostable or bioabsorbable polymer or combinations thereof with the therapeutic agents incorporated therein. Typical material properties for coatings include flexibility, ductility, tackiness, durability, adhesion and cohesion. Biostable and bioabsorbable polymers that exhibit these desired properties include methacrylates, polyurethanes, silicones, poly (vinyl acetate), poly (vinyl alcohol), ethylene vinyl alcohol, poly (vinylidene fluoride), poly (lactic acid), poly (glycolic acid), poly (caprolactone), poly (trimethylene carbonate), poly (dioxanone), polyorthoester, polyanhydrides, polyphosphoester, polyaminoacids as well as their copolymers and blends thereof.

As described above, polymer stents may contain therapeutic agents as a coating, e.g. a surface modification. Alternatively, the therapeutic agents may be incorporated into the stent structure, e.g. a bulk modification that may not require a coating. For stents prepared from biostable and/or bioabsorbable polymers, the coating, if used, could be either biostable or bioabsorbable. However, as stated above, no coating may be necessary because the device itself is fabricated from a delivery depot. This embodiment offers a number of advantages. For example, higher concentrations of the therapeutic agent or agents may be achievable such as about >50 percent by weight. In addition, with higher concentrations of therapeutic agent or agents, regional drug delivery (>5 mm) is achievable for greater durations of time. This can treat different lesions such as diffused lesions, bifurcated lesions, small and tortuous vessels, and vulnerable plaque. Since these drug loaded stents or other devices have very low deployment pressures (3 to 12 atmospheres), it will not injure the diseased vessels. These drug-loaded stents can be delivered by different delivery systems such balloon expandable; self-expandable or balloon assist self-expanding systems.

Although the present invention has been described above with respect to particular preferred embodiments, it will be apparent to those skilled in the art that numerous modifications and variations can be made to these designs without departing from the spirit or essential attributes of the present invention. Accordingly, reference should be made to the appended claims, rather than to the foregoing specification, as indicating the scope of the invention. The descriptions provided are for illustrative purposes and are not intended to limit the invention nor are they intended in any way to restrict the scope, field of use or constitute any manifest words of exclusion.

What is claimed is:

1. An apparatus comprising:
a structure constructed from at least one polymer, said structure being capable of implantation into an anatomical conduit; and
wherein the at least one polymer is prepared such that it exhibits a morphological condition whereby the structure maintains a pre-determined form when implanted into the anatomical conduit.

2. The apparatus of claim 1, wherein the at least one polymer comprises bioabsorbable polymers.

3. The apparatus of claim 1, wherein the bioabsorbable polymer comprises poly (alpha hydroxy esters).

4. The apparatus of claim 1, wherein the polymer is prepared such that the morphological condition of the polymer has a crystallinity ranging from 0-50%.

5. The apparatus of claim 1 further comprising a therapeutic agent dispersed throughout the polymer wherein the preparation of the polymer to obtain the optimal crystallinity allows the therapeutic agent to remain substantially crystalline.

6. The apparatus of claim 1, further comprising at least one therapeutic agent dispersed throughout the at least one polymer in a concentration of up to thirty percent.

7. The apparatus of claim 1, wherein the at least one polymer comprises a blend of one or more polymers.

8. The apparatus of claim 1, wherein the at least one polymer comprises a blend of at least one polymer and at least one plasticizer.

9. The apparatus of claim 1, wherein the structure comprises a stent.

10. The apparatus of claim 1, wherein the at least one polymer comprises non-bioabsorbable polymers.

11. The apparatus of claim 10, wherein the non-bioabsorbable polymer comprises polyurethane.

12. The apparatus of claim 1, wherein the structure comprises a covered stent.

13. The apparatus of claim 6, wherein the at least one therapeutic agent comprises anti-proliferative agents.

14. The apparatus of claim 6, wherein the at least one therapeutic agent comprises anti-thrombogenic agents.

15. The apparatus of claim 6, wherein the at least one therapeutic agent comprises anti-restenotic agents.

16. The apparatus of claim 6, wherein the at least one therapeutic agent comprises anti-infective agents.

17. The apparatus of claim 6, wherein the at least one therapeutic agent comprises anti-viral agents

18. The apparatus of claim 6, wherein the at least one therapeutic agent comprises anti-bacterial agents.

19. The apparatus of claim 6, wherein the at least one therapeutic agent comprises anti-fungal agents.

20. The apparatus of claim 6, wherein the at least one therapeutic agent comprises anti-inflammatory agents

21. The apparatus of claim 6, wherein the at least one therapeutic agent comprises cytostatic agents.

22. The apparatus of claim 6, wherein the at least one therapeutic agent comprises cytotoxic agents.

23. The apparatus of claim 6, wherein the at least one therapeutic agent comprises immunosuppressive agents

24. The apparatus of claim 6, wherein the at least one therapeutic agent comprises anti-microbial agents.

25. The apparatus of claim 6, wherein the at least one therapeutic agent comprises anti-calcification agents.

26. The apparatus of claim 6, wherein the at least one therapeutic agent comprises anti-encrustation agents.

27. The apparatus of claim 6, wherein the at least one therapeutic agent comprises statins.

28. The apparatus of claim 6 wherein the at least one therapeutic agent comprises hormones.

29. The apparatus of claim 6, wherein the at least one therapeutic agent comprises anti-cancer agents.

30. The apparatus of claim 6, wherein the at least one therapeutic agent comprises anti-coagulants.

31. The apparatus of claim 6, wherein the at least one therapeutic agent comprises anti-migratory agents.

32. The apparatus of claim 6, wherein the at least one therapeutic agent comprises tissue growth promoting agents.

33. The apparatus of claim 1, wherein the structure comprises a heparin coated stent.

34. The apparatus of claim 1 further comprising a radiopaque material dispersed throughout the structure.

35. The apparatus of claim 1 wherein the structure is coated with a radiopaque material.

36. An apparatus comprising:
a structure formed from a first polymeric material; and
a coating layer affixed to the first polymeric material,
wherein the first polymeric material is prepared such that it exhibits a morphological condition that allows the structure to maintain a desired condition.

37. The apparatus of claim 36 wherein the coating layer comprises at least one therapeutic agent dispersed throughout a second polymeric material in a concentration of up to thirty percent.

38. The apparatus of claim 36, wherein the structure comprises a stent.

39. The apparatus of claim 36, wherein the first polymer comprises a bioabsorbable polymer.

40. The apparatus of claim 36, wherein the first polymer comprises non-bioabsorbable polymer.

41. The apparatus of claim 39, wherein the bioabsorbable polymer comprises poly (alpha hydroxy esters).

42. The apparatus of claim 40, wherein the non-bioabsorbable polymer comprises polyurethane.

43. The apparatus of claim 36, wherein the structure comprises a covered stent.

44. The apparatus of claim 36 further comprising a therapeutic agent.

45. The apparatus of claim 36, wherein the polymer is prepared such that the morphological condition of the polymer has a crystallinity ranging from 0-50%.

46. The apparatus of claim 45, wherein the morphological condition of the polymer resists recoil allowing the structure to maintain the desired condition.

47. The apparatus of claim 44 wherein the therapeutic agent is dispersed throughout the first polymeric material wherein the first polymeric material is prepared to obtain a desired semi-amorphous structure while the therapeutic agent remains substantially crystalline.

48. The apparatus of claim 36 wherein the first polymeric material comprises a blend of polymers.

50. An apparatus comprising:
a structure constructed from at least one polymer, said structure being capable of implantation into an anatomical conduit;
a therapeutic agent that is delivered from the structure;
wherein the at least one polymer is prepared such that it exhibits an optimal morphological condition whereby the structure maintains a pre-determined form when implanted into the anatomical conduit.

51. The apparatus of claim 50 wherein the therapeutic agent is dispersed throughout the structure.

52. The apparatus of claim 50 wherein the structure is coated with the therapeutic agent.

53. The apparatus of claim 50, wherein the structure comprises a bifurcated stent.

54. The apparatus of claim 50, wherein the structure comprises a stent.

55. The apparatus of claim 50, wherein the structure comprises a vascular filter.

56. The apparatus of claim 50, wherein the structure comprises an aneurismal repair device.

57. The apparatus of claim 50, wherein the structure comprises devices for treating diffuse arterial lesions.

58. The apparatus of claim 50, wherein the structure comprises devices for treating superficial femoral artery disease.

59. The apparatus of claim 50, wherein the structure comprises devices for treating below the knee arterial disease.

60. The apparatus of claim 50, wherein the structure comprises venous valves.

61. The apparatus of claim 50, wherein the structure comprises heart valves.

62. The apparatus of claim 50, wherein regional delivery includes elution of the at least one therapeutic agent upstream of the structure.

63. The apparatus of claim 50, wherein regional delivery includes elution of the at least one therapeutic agent downstream of the structure.

64. The apparatus of claim 50, wherein regional delivery includes elution of the at least one therapeutic agent to an adjacent vessel proximate to the structure.

65. The apparatus of claim 50, wherein regional delivery includes elution of the at least one therapeutic agent to an organ proximate to the structure.

66. The apparatus of claim 50, further comprising a radiopaque agent.

67. The apparatus of claim 50, wherein the structure comprises devices for treating smaller vessels.

68. The apparatus of claim 50, wherein the structure comprises devices for treating tapered vessels.

69. The apparatus of claim 50, wherein the structure comprises devices for treating tortuous vessels
